(19) United States
(12) Patent Application Publication
(10) Pub. No.: US 2011/0196478 A1
(43) Pub. Date: Aug. 11, 2011

(54) DEVICES AND METHODS FOR LUMEN TREATMENT

(75) Inventor: Mikhail T. Torosoff, Loudonville, NY (US)

(73) Assignee: Beoptima Inc.

(21) Appl. No.: 13/024,981

(22) Filed: Feb. 10, 2011

Related U.S. Application Data
(60) Provisional application No. 61/303,102, filed on Feb. 10, 2010.

(51) Int. Cl. A61F 2/82

(52) U.S. Cl. 623/1.36

(57) ABSTRACT

Devices and methods for lumen treatment are provided. According to aspects illustrated herein, there is provided an endoprosthesis that includes an internal layer designed to provide a negative electric field directed endoluminally; an external layer designed to provide a positive electric field directed exoluminally; and one or more intermediate layers disposed between the internal layer and the external layer, wherein the negative electric field is due to a negative point charge between about −25 mV and about −250 mV, and wherein the positive electric field is due to a positive point charge between about +1 mV and about +30 mV.

### RELATED APPLICATIONS

This application claims the benefit of and priority to U.S. Provisional Patent Application No. 61/303,102, filed on Feb. 10, 2010, the entirety of this application is hereby incorporated herein by reference for the teachings therein.

### FIELD

The embodiments disclosed herein relate to devices and methods for lumen treatment, and more particularly to endoprosthesis sufficiently designed to provide both a negative electric field and a positive electric field.

### BACKGROUND

Stent implantation has become a preferred treatment method for obstructive vascular lesions, such as atherosclerotic plaques and fibromuscular dysplasia, as well as for aneurysmal vascular lesions, such as vein graft lesions and Kawasaki disease. Clinical outcomes of stenting are plagued by stent thrombosis and in-stent re-stenosis. Luminal stent thrombosis has been shown to be mediated by fibrin and platelet deposition, while in-stent re-stenosis is typically a result of neointimal hyperplasia proceeding from the vessel wall towards the vessel lumen.

### SUMMARY

Devices and methods for lumen treatment are provided. According to aspects illustrated herein, there is provided an endoprosthesis that includes an internal layer designed to provide a negative electric field directed endoluminally; an external layer designed to provide a positive electric field directed exoluminally; and one or more intermediate layers disposed between the internal layer and the external layer, wherein the negative electric field is due to a negative point charge between about −25 mV and about −250 mV, and wherein the positive electric field is due to a positive point charge between about +1 mV and about +30 mV.

According to aspects illustrated herein, there is provided an endoprosthesis that includes a plurality of struts, wherein each strut has an internal layer, an external layer, and one or more intermediate layers therebetween, wherein the internal layer includes a material that provides a negative electric field directed endoluminally, wherein the external layer includes a material that provides a positive electric field directed exoluminally, and wherein the one or more intermediate layers include a material that provides an insulation between the internal layer and the external layer.

According to aspects illustrated herein, there is provided a method of treating a blood vessel that includes deploying an endoprosthesis inside the blood vessel, the endoprosthesis comprising: an internal layer designed to provide a negative electric field directed endoluminally; an external layer designed to provide a positive electric field directed exoluminally; and one or more intermediate layers disposed between the internal layer and the external layer, wherein the negative electric field is created by a negative point charge between about −25 mV and about −250 mV, and wherein the positive electric field is created by a positive point charge between about +1 mV and about +30 mV so as to treat the blood vessel.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 is a longitudinal cross-sectional schematic view of a blood vessel showing the three layers of the blood vessel.

FIGS. 2A-2D show an embodiment of an endoprosthesis of the present disclosure.

FIGS. 3A-3F show various embodiments of struts of an embodiment of an endoprosthesis of the present disclosure.

FIG. 4A and FIG. 4B show schematic illustrations of an embodiment of an endoprosthesis of the present disclosure. FIG. 4A is a schematic representation of a cross-section of an individual strut positioned at a vessel wall and lumen interface with positive electric field directed outwards, towards the vessel wall, and negative electric field directed endoluminally. FIG. 4B is a schematic illustration of the endoprosthesis cross-section with plurality of struts creating overlapping electric fields of desired polarity.

FIG. 5A and FIG. 5B illustrate a schematic illustration of distribution of electric charge in an embodiment of an endoprosthesis of the present disclosure.

FIG. 6 shows a schematic sectional illustration of an embodiment of an endoprosthesis of the present disclosure.

FIGS. 7A-7C show some of the method steps for utilizing an embodiment of an endoprosthesis of the present disclosure. FIG. 7A and FIG. 7B show the endoprosthesis being positioned in a body lumen. FIG. 7C shows an endoprosthesis positioned in the body lumen.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The instant disclosure provides an endoprosthesis capable of producing electric fields of different polarity, intensity and direction. In particular, the endoprosthesis of the present disclosure is designed to provide a negative electric field that is directed endoluminally and is sufficient to reduce stent thrombosis and in-stent re-stenosis, while, at the same time, providing a positive electric field that is directed exoluminally and is sufficient to promote secure anchoring of the endoprosthesis in situ. In an embodiment, the endoprosthesis of the present disclosure is designed, so as to minimize the spread of the negative electric field exoluminally, while also minimizing the spread of the positive electric field endoluminally. In other words, the negative electric field is substantially contained in the endoluminal region of the endoprosthesis and the positive electric field is substantially contained in the exoluminal region.

As used herein, the term “endoprosthesis” include, but are not limited to, stents and stent-grafts. Endoprosthesis of the present disclosure include, but are not limited to, vascular endoprosthesis, urethral endoprosthesis, esophageal endoprosthesis, digestive tract endoprosthesis, and biliary endoprosthesis.

As used herein, the term “lumen” refers to the channel within a tubular structure such as a blood vessel or a stent, or to the cavity within a hollow organ such as the intestine or the urethra. “Intraluminal” means inside the lumen.

As used herein, the term “negative electric field” refers to an electric field created by a negative charge.

As used herein, the term “positive electric field” refers to an electric field created by a positive charge.

As used herein, the term “restenosis” means the reoccurrence of stenosis, a narrowing of a blood vessel, leading to restricted blood flow. Restenosis usually pertains to an artery or other large blood vessel that has become narrowed, received treatment to clear the blockage and subsequently become renarrowed. This is usually restenosis of an artery, or other blood vessel, or possibly a vessel within an organ. Damage to the blood vessel wall by angioplasty triggers physiological response that can be divided into two stages. The first stage that occurs immediately after tissue trauma, is thrombosis. A blood clot forms at the site of damage and further hinders blood flow. This is accompanied by an inflammatory immune response. The second stage tends to occur 3-6 months after surgery and is the result of proliferation of cells in the intima, a smooth muscle wall in the vessel. This is also known as Neointimal hyperplasia (NIHA). Although the use of stents has limited the incidence of restenosis, in-stent restenosis remains an important problem.

As used herein, the term “stent” refers to a generally tubular article for implantation into a body lumen.

As used herein, the term “stent graft” refers to a tube comprising fabric supported by a stent.

As used herein, the term “strut” means a structural member of an endoprosthesis of the present disclosure. In an embodiment, the strut acts as a support layer of an endoprosthesis of the present disclosure.

As used herein, the term “thrombosis” refers to the formation of a blood clot (thrombus) inside a blood vessel, obstructing the flow of blood through the circulatory system. Stent thrombosis is a rare complication following stent implantation; if thrombosis occurs, however, thrombosis is associated with a high morbidity and mortality.

As used herein, the term “endoluminally” means away from a lumen wall.

As used herein, the term “exoluminally” means toward a lumen wall.

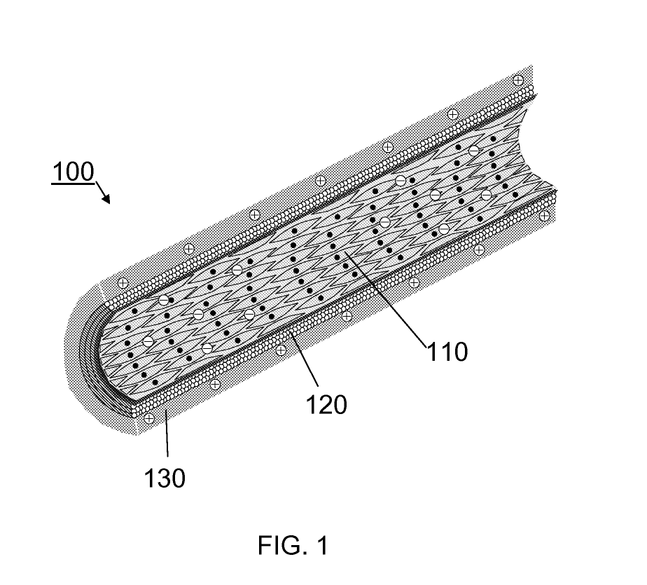

FIG. 1 is a longitudinal cross-sectional schematic view of a blood vessel 100, such as an artery or a vein. The blood vessel 100 has three layers, from inside to outside: Tunica intima 110 (the thinnest layer), which is a single layer of endothelial cells (endothelium) glued by a polysaccharide intercellular matrix, surrounded by a thin layer of subendothelial connective tissue interlaced with a number of circularly arranged elastic bands called the internal elastic lamina; Tunica media 120 (the thickest layer), which includes circularly arranged elastic fiber, connective tissue, polysaccharide substances, the second and third layer are separated by another thick elastic band called external elastic lamina; and the Tunica adventitia 130, which is entirely made of connective tissue. The normal, non-atherosclerotic endothelium 110 has an intraluminal negative electric charge between about −0.5 mV to about −120 mV; about −5.0 mV to about −42 mV. It is believed that prostaglandins and cialic acid are predominantly responsible for maintaining this endothelial negative electric charge. Intraluminal endothelial negative electric charge has been shown to prevent adhesion of the similarly charged monocytes and platelets to the vessel wall. Treatment with aspirin, known to prevent platelet aggregation, increases negative electric charge of platelets. Conversely, decrease in negative electric charge promotes platelet aggregation, fibrin aggregation, and thrombus formation. The tunica adventitia 130 possesses a net positive charge relative to the endothelium 110.

Atherosclerosis (also known as Arteriosclerotic Vascular Disease or ASVD) is the condition in which an artery wall thickens as the result of a build-up of fatty materials such as cholesterol. Atherosclerosis is commonly referred to as a hardening or furring of the arteries, and is caused by the formation of multiple plaques within the arteries. Atherosclerotic endothelium has been shown to decrease the endothelial negative electric charge. Stents are commonly used to counter narrowing of arteries due to plaque deposition and hardening. Existing stent designs have solved the task of restoring vessel (cavity) geometry but failed to address problems of instent thrombosis and neointimal hyperplasia.

It has been discovered that stent thrombosis and platelet aggregation are at least in part mediated by electric charge phenomena and may be counteracted by creating negative luminal electric charge. The same negative electric charge directed alongside the vessel appears to prevent neointimal formation and promote stent endothelization. Some neointimal formation, however, is beneficial and has been found to be advantageous for secure stent anchoring onto the vessel wall. When negative charge is directed towards the endothelium of the vessel wall, poor strut anchoring may lead to stent migration. Conversely, limited positive charge directed towards the endothelium of the vessel wall may promote cell proliferation and ensure lasting stent anchoring. However, when only positive charge is present, excessive neointimal hyperplasia may promote in-stent re-stenosis.

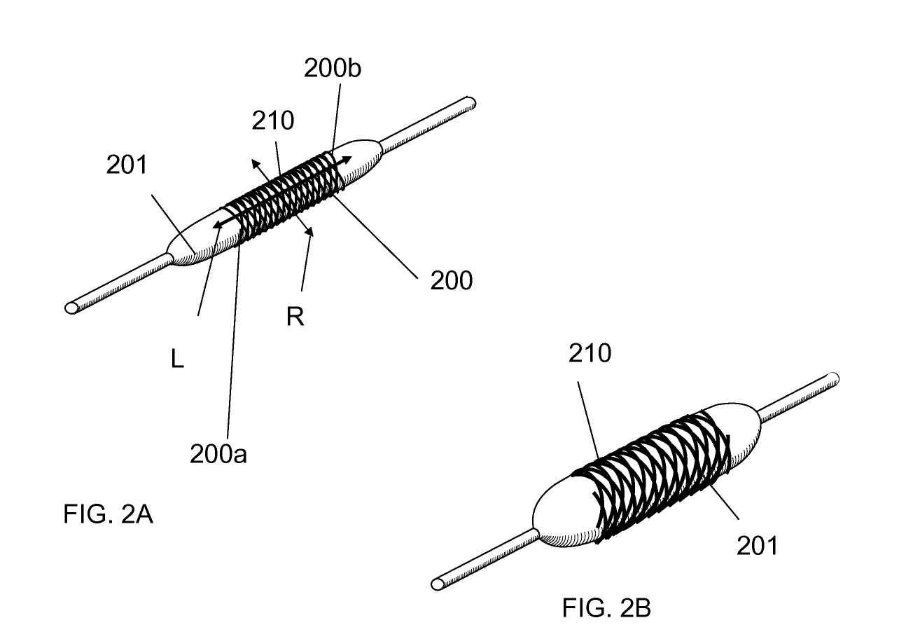

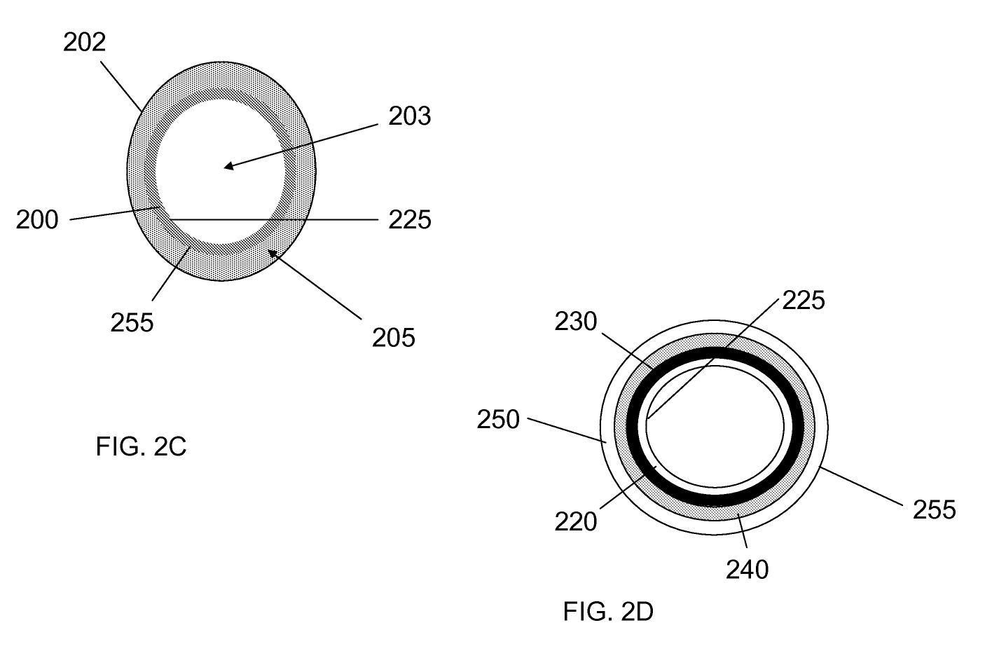

FIGS. 2A-2B illustrate an embodiment of an endoprosthesis 200 of the present disclosure disposed over a balloon 201. The endoprosthesis 200 includes a first free margin 200a and a second free margin 200b. The endoprosthesis 200 has a longitudinal direction, generally indicated by the arrow L, and a radial direction, generally indicated by the arrow R. FIG. 2A illustrates the endoprosthesis 200 in an unexpanded configuration having a reduced cross-section. On the other hand, FIG. 2B illustrates the endoprosthesis 200 in an expanded configuration having an increased cross-section. Although illustrated as being expanded by the balloon 201, the endoprosthesis 200 may be self-expandable. In an embodiment, the endoprosthesis 200 includes a plurality of struts 210 designed to provide structural support to the endoprosthesis 200. The struts 210 may be of any shape, including, but not limited to, u-shaped, v-shaped, spiral-shaped, w-shaped, straight, n-shaped, z-shaped, and the like. The cross-section of the struts 210 may also vary so as to impart the endoprosthesis 200 with desired characteristics.

FIG. 2C show the endoprosthesis 200 implanted into a lumen 202. The endoprosthesis 200 includes an inner surface 225 that defines an endoluminal region 203 and an outer surface 255 that faces an exoluminal region 205. In reference to FIG. 2D, the endoprosthesis 200 comprises multiple layers: an internal or endoluminal layer 220, an external or exoluminal layer 250, and one or more intermediate layers 230, 240 disposed between the internal layer 220 and the external layer 250.

Each layer 220, 230, 240, and 250 of the endoprosthesis 200 may comprise a single material, alloy, or have a combination of materials which may be layered, interwoven, and/or arranged in any other way or fashion. In an embodiment, each layer 220, 230, 240, and 250 of the endoprosthesis 200 may comprise, independently of other layers, multiple sub-layers. The layers 220-250 may have, independently of other layers, any design conventionally used in the art for endoprosthesis. The layers 220-250 may have the same shape or different shapes.

In an embodiment, the endoprosthesis 200 includes a stent having a plurality of struts and having an inner surface and an outer surface. The stent may serve as a structural layer for providing structural support to the endoprosthesis 200. Any conventionally known stent design may be utilized. The internal layer 220 may comprise a first material disposed along the inner surface of the stent and adapted to provide a negative electric charge directed endoluminally. The external layer 250 may comprise a second material disposed along the outer surface of the stent and adapted to provide a positive electric charge directed exoluminally. Additionally, the one or more intermediate layers may include an insulating layer formed with an insulating material between the internal layer 220 and the external layer 250. In an embodiment, the stent may be of a material that can provide a negative electric charge directed endoluminally, can provide a positive electric charge directed exoluminally, can serve as an insulator, or a combination thereof, and thus the endoprosthesis 200 may not need a separate structural layer.

In an embodiment, the shape of the layers 220-250 determines the shape of the inner and outer surfaces 225, 255 of the endoprosthesis 200. The inner and outer surfaces 225, 255 of the endoprosthesis 200 may be provided with any shape, as desired. The inner and outer surfaces 225, 255 may have the same shape or different shapes. In an embodiment, the shape of the inner and outer surface may be varied by varying the thickness of the internal layer and the external layer, respectively, longitudinally from the first free margin 200a to the second free margin 200b of the endoprosthesis 200 to the opposite side. For example, the internal and external layer may, independently of each other, 1) have a constant thickness from the first free margin 200a to the second free margin 200b, 2) have an increased thickness at the first and second free margins 200a and 200b relative to the thickness in the middle region of the layer; or 3) have a decreased thickness at the first and second free margins 200a and 200b relative to the middle region of the layer.

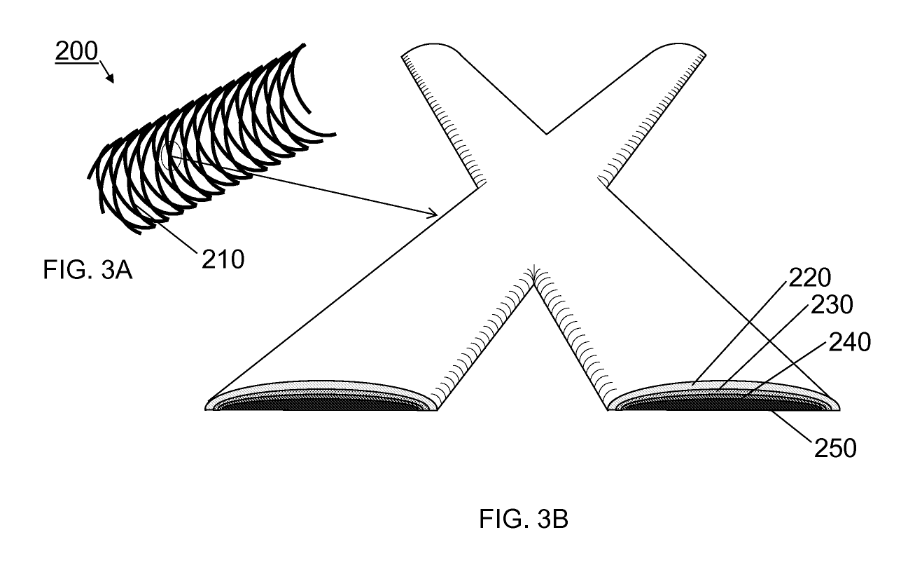

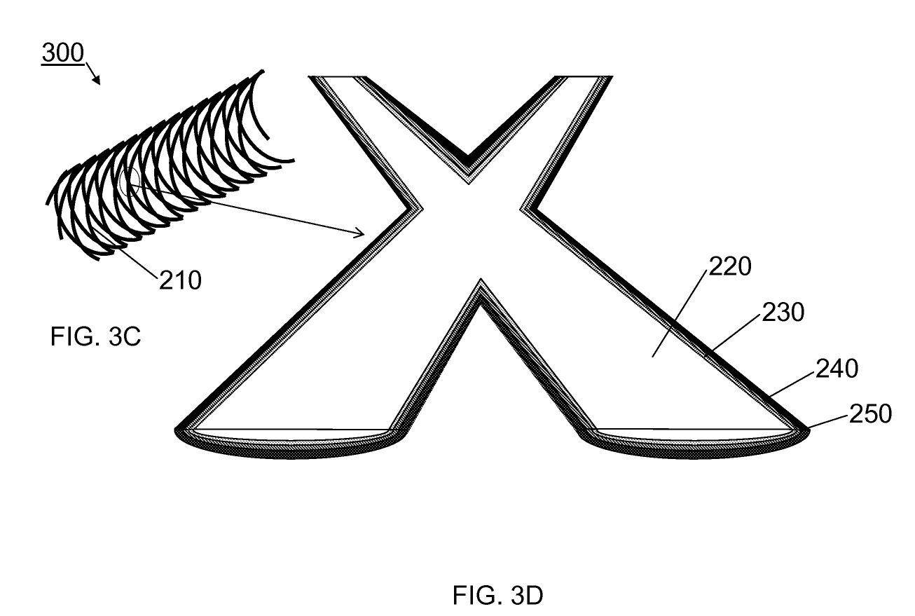

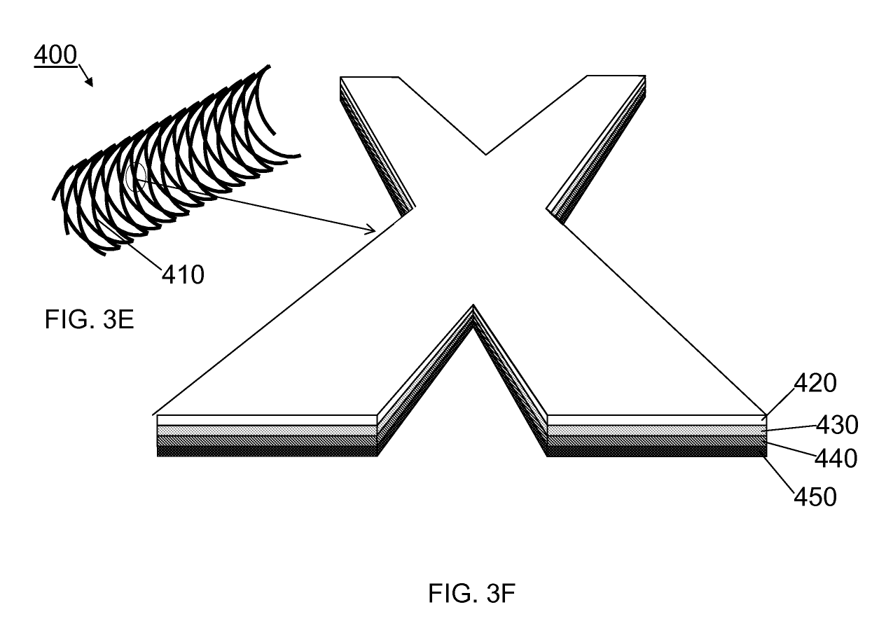

By way of a non-limiting example, FIGS. 3A-3B illustrate an embodiment of the endoprosthesis 200 where the internal layer 220 convex endoluminaly, and thus the inner surface 225 is also convex. In another embodiment, as illustrated in FIGS. 3C-3D, the external layer 250 is convex exoluminaly, and thus the outer surface 255 is convex. In yet another embodiment, as illustrated in FIGS. 3E-3F, the internal layer 220 and the external layer 250 are both flat, and so are the inner surface 225 and the outer surface 255. In an embodiment, the layers may all have substantially the same shape. For example, in an embodiment with the structural layer comprising a stent, the stent may have flat struts and the other layers may be formed over the struts, in such a manner that the inner surface 225 and the outer surface 255 are flat. In another embodiment, the layers 220-250 may have different shapes. For example, in an embodiment with the structural layer comprising a stent, the stent may have flat struts and the other layers may be formed over the struts, so as to provide the inner surface 225, the outer surface 255, or both with a shape other than flat.

In an embodiment, the endoprosthesis 200 may have an overall thickness, including all layers, of about 200 μm or less. In an embodiment, the endoprosthesis 200 may have an overall thickness, including all layers between about 100 μm and about 300 μm. In an embodiment, the internal layer 220 may have a thickness of between about 30 μm and about 150 μm. In an embodiment, the internal layer 220 may have a thickness between about 40 μm and about 50 μm. In an embodiment, the external layer 250 may have a thickness less than about 100 μm. In an embodiment, the external layer 250 may have a thickness between about 20 μm and 30 μm. In an embodiment, the one or more intermediate layers 230, 240 may include an insulating material having a thickness of between about 10 μm and about 50 μm. In some embodiments, the thicknesses of the internal layer 220 and/or the external layer 250 may depend on the type of respective materials used to form these layers 220, 250, as well as on the desired duration, intensity, and relative contributions of negative and positive electric fields provided by the layers, as is described in detail below.

In an embodiment, the internal layer 220 is sufficiently designed so that the internal layer 220 counteracts aggregation of blood cells and/or proteins on the inner surface 225 of the endoprosthesis 200. In an embodiment, the external layer 250 is sufficiently designed, so that the external layer 250 promotes secure anchoring of the endoprosthesis 200 to a lumen into which the endoprosthesis 200 is implanted. In an embodiment, such secure anchoring of the endoprosthesis 200 may be achieved by promoting cell adhesion to the endoprosthesis 200. In an embodiment, the endoprosthesis 200 is sufficiently designed to provide electric fields of different polarity, intensity and direction. For example, the internal layer 220 may be adapted to provide a negative electric field directed substantially endoluminally, while the external layer 250 may, in embodiment, be adapted to provide a positive electric field directed substantially exoluminally.

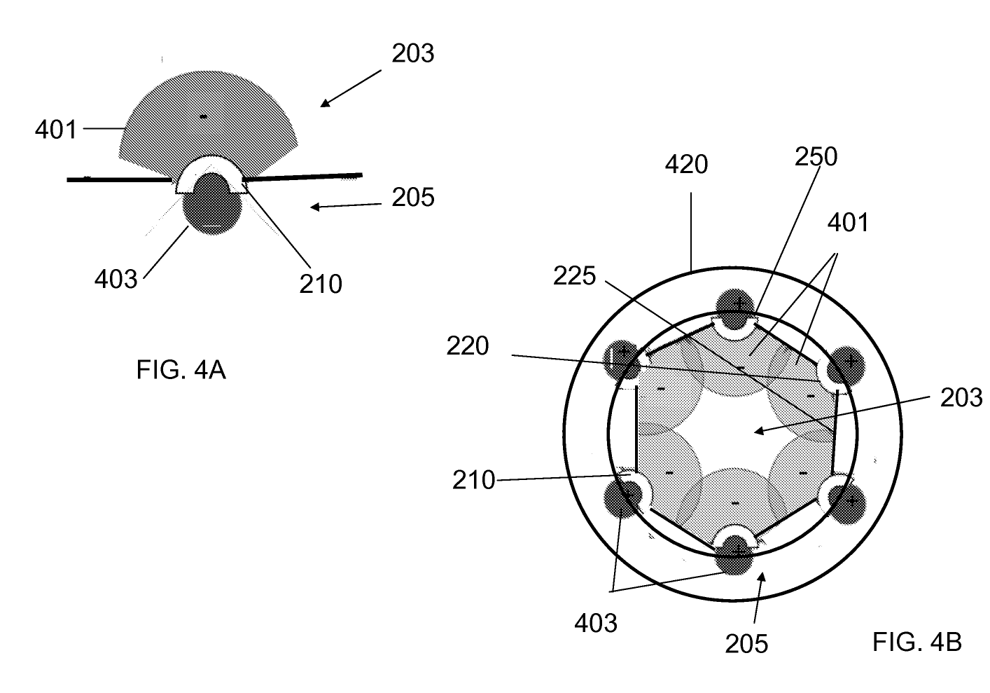

In reference to FIG. 4A, in an embodiment, the internal layer 220 may be designed to provide an inward negative electric field 401 directed into the endoluminal region 203. Such negative electric field may be less than about 180 degrees. In another embodiment, the internal layer 220 may be designed to provide an inward negative electric field 401 between about 120 and 150 degrees. In yet another embodiment, the internal layer 220 may be designed to provide an inward negative electric field 401 of about 150 degrees. In an embodiment, the external layer 250 may be designed such that the struts 210 provide an outward positive electric field 403 directed into the exoluminal region 205. In an embodiment, the outward positive electric field 403 may be less than about 150 degrees. In another embodiment, the external layer 250 may be designed such that the outward positive electric field 403 less than about 120 degrees. In yet another embodiment, the external layer 250 may be designed such that the outward positive electric field 403 is between about 60 and about 90 degrees. As illustrated in FIG. 4A, by way of a non-limiting example, the inner surface 225 of the endoprosthesis 200 is convex and the outer surface 255 is concave.

In one embodiment, the design of a charged layer, i.e. the internal layer 220 or external layer 250, and thus the shape of the electric field created by the charged layer, may be varied by varying the shape of the charged layer; a material forming the layer; the distribution of the material in the charged layer; the shape, thickness or both of the one or more intermediate layers; or combinations thereof. In addition, it should be noted that, in some embodiments, the shape and/or strength of the electric field provided by a charged layer may be altered by providing a counteracting electric field of an opposite sign. Accordingly, in an embodiment, the combined design of the endoprosthesis 200 is such that the internal layer is capable of providing the negative electric field 401, as described above, while, at the same time, the external layer is capable of providing the positive electric field 403, as described above. In an embodiment, the designs of the internal layer 220, the external layer 250, and the one or more intermediate layers 230, 240, are such that the total negative electric field and the total positive electric field interact to result in the negative electric field 401 and the positive electric field 403, as described above.

Referring now to FIG. 4B, which shows the endoprosthesis 200 implanted into a lumen 420, in an embodiment, the endoprosthesis 200 is designed such that the negative electric fields 401 created by the internal layer 220 overlap to blanket substantially the entire inside surface 225 of the endoprosthesis 200. That is, a minimum desired surface charge is maintained along the inner surface of the endoprosthesis 200. In an embodiment, such minimum desired surface charge is between about −25 mV to about −200 mV. As illustrated in FIG. 4B, the internal layer 220 is designed such that the negative electric field 401 from each strut 210 is dispersed both in the radial direction and the longitudinal direction. In this manner, the negative electric fields 401 produced by various struts 210 overlap, so that the entire inside surface 225 of the endoprosthesis 200 is covered with the negative electric field. Moreover, in an embodiment, the external layer 250 of the endoprosthesis 200 is shaped such that the positive electric fields 403 are substantially limited to the struts 210 of the endoprosthesis 200. That is, the external layer 250 is designed such that the positive electric field 403 from an individual strut 210 is dispersed exoluminally in the radial direction, while the dispersion of the positive electric field 403 exoluminally in the longitudinal direction is minimized or eliminated. In addition, the internal layer 220 and the external layer 250 are shaped such that the negative electric fields 405 are substantially contained in the endoluminal region 203 and the positive electric fields 407 are substantially contained in the exoluminal region 205. The term “substantially contained” as used herein means the electric field outside the region in which the field is contained is so small that is effectively has no effect on the particles in or adjacent outside the region in which the field is contained.

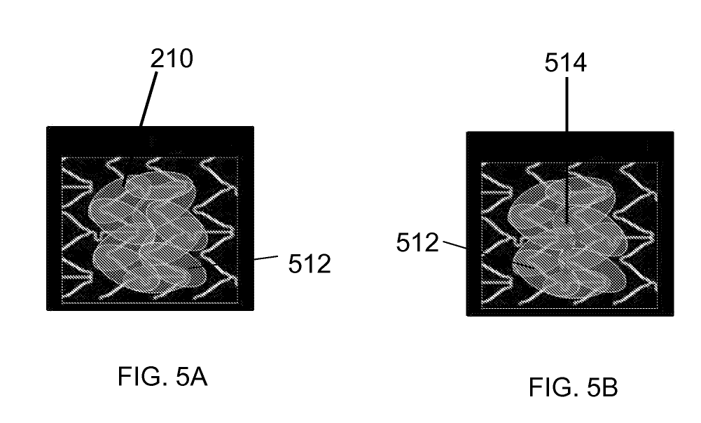

As generally illustrated in FIG. 5A, each strut 210 of the endoprosthesis 200 can produce an electric field 512, due to a charged material disposed on the strut. In an embodiment, the smallest amount of electric charge, or an mount of electric force acting on a particle, may generally exist at overlapping points 514, where electric fields produced by adjacent struts overlap, as generally illustrated by the stars in FIG. 5B. In an embodiment, the endoprosthesis 200 may be provided with a design, such that the charge at overlapping points 514 is approximately about −25 mV to about−200 mV.

As noted above, in an embodiment, the endoprosthesis 200 of the present disclosure comprises a stent having an inner surface, an outer surface, and a plurality of struts, the internal layer 220 comprising a first material capable of generating a target negative electric charge disposed on the struts along the inner surface of the stent, the external layer 250 comprising a second material capable of generating a target positive electric charge disposed on the struts along the outer surface of the stent and an insulating layer 230 disposed between the internal layer 220 and the external layer 250. Accordingly, in an embodiment, the first material and the second material disposed on a strut may create a negative point charge on the strut and the second material may create a positive point charge, respectively. Because the charge at a point of interest away from a strut is directly proportional to the magnitude of a point charge on the strut and inversely proportional to the distance between the point of interest and the strut, a desired magnitude of the point charge can be calculated. The desired magnitude of a point charge may be achieved through material selection, by varying the shape of a material disposed on the struts, by varying the amount of a material disposed on the struts, or a combination thereof. In an embodiment, the negative point charge at the struts may be between about −150 mV and about −250 mV. In another embodiment, the negative point charge may be between about −175 mV and about −200 mV. On the other hand, in an embodiment, the positive point charge may vary from about +1 mV to about +30 mV. In another embodiment, the positive point charge may vary from about +1 mV to about +10 mV. In yet another embodiment, the positive point charge may from about 20 mV to about 100 mV.

In an embodiment, an isotope may be utilized to provide a negative or a positive charge. For example, the first material may comprise an isotope that can emit beta rays, which carry a negative charge. In such an embodiment, the point charges are not limited to the surface of the struts 210, but rather is distributed throughout the area of the negative electric field 512. Again, because the charge at a point of interest is inversely proportional to the distance between the point of interest and the location of the point charge, the point charges may thus be smaller in this embodiment, than in an embodiment, where the point charge is limited to the surface of the struts. In an embodiment, the point charge may range between about −25 mV to about −200 mV.

The insulating layer 230 may be designed such that the negative point charges are separated from the positive point charges. In this manner, the negative charges and the positive charges may remain on the inner surface of the stent and the outer surface of the stent, respectively. In an embodiment, such as when an isotope is used, the insulating layer may be designed such as to provide a shield for the preferential prevention of outward negative charge and preferential prevention of the inward positive charge.

The internal layer 220 and the external layer 250 may comprise any material capable of providing a target negative charge and a target positive charge, respectively, for a desired duration of time, when the endoprosthesis 200 is exposed to a physiological fluid, such as blood. In an embodiment, the one or more materials selected for the internal layer 220 may be capable of maintaining the target negative charge for at least 6 months. In an embodiment, the negative charge may be provided for between about 6 weeks and about 6 months. In an embodiment, the internal layer 220 may comprise a metallic material, such as titanium, aluminum, cobalt, or any other metal or alloy material with similar properties. Some of the advantages associated with using solid metals for the negative and positive electric charge materials are ease of manufacturing and constant and predictable amount of charge associated with the material. Some possible disadvantages are the amplitude of the charge which is limited by the intrinsic properties of the selected material. In an embodiment, the internal layer 220 may comprise an isotope capable of providing the target negative electric charge. The amount of charge associated with radioactive emitters may be manipulated by isotope selection and adjusting thickness of the deposited layer. However, emissions may diminish overtime, which may not occur with a charge of the metals. In another embodiment, the internal layer 220 may comprises a polymer capable of providing the target negative electric charge. Polymers are an attractive option considering ease of application and plasticity. Limitations of polymers are concerned with intrinsic electric properties and thickness of material required to create a required electromagnetic charge.

In an embodiment, the internal layer 220 may comprise one or more materials capable of producing the target negative charge, as described above, when exposed to a physiological fluid, such as blood. As noted above, in an embodiment, the type of material and the thickness of the material may be varied to achieve the target negative charge. In an embodiment, the internal layer 220 may comprise a metallic material, such as titanium, aluminum, cobalt, or any other metal or alloy material with similar properties. These materials, when exposed to blood electrolytes, are capable of producing and sustaining a negative electric charge of −250 mV for at least about 6 weeks. In another embodiment, the internal layer 220 may comprises a polymer capable of providing the target negative electric charge. Examples of such polar polymers include, but are not limited to, polyvinylidene fluoride, polyvinylidene chloride, silicone rubber, polyethylene, polyvinyl chloride, polyurethane, polypropylene, teflon, cellulose, acrylic resins, polyarylates (L-tyrosine-derived), free acid polyarylates, polycarbonates (L-tyrosine-derived), polyester-amides), polypropylene fumarate-co-ethylene glycol) copolymer, polyanhydride esters, polyanhydrides, polyorthoesters, silk-elastin polymers, copolymers of said polymers and mixtures thereof. In yet another embodiment, the internal layer 220 may any other biologically compatible material capable of providing the target negative electric charge. Examples of such polar compounds include, but are not limited to nitric oxide, heparin, heparin derivatives, other anti-coagulation factors, hyaluronic acid, prostaglandins, cialic acid, derivates and mixtures thereof.

In an embodiment, the internal layer 220 may comprise an isotope capable of providing the target negative electric charge. In an embodiment, a beta-minus emitting isotope may be deposited onto an outer wall surface of a endoprosthesis strut. Any such isotope capable of providing the target negative charge for at least 6 months may be utilized. In an embodiment, the negative charge may be provided for between about 6 weeks and about 6 months. Examples of such isotopes include, but are not limited to, Cerium-141; Gallium-67; Gold-198; Iridium-192, Iron-59, Iodine-123, I-125, I-126, I-131; Indium-111; Nickel-63; Phosphorus-32; Promethium-147; Rhenium-186; Ruthenium-103; Samarium-153; Selenium-75; Silver-111; Strnotium-82, Sr-85, Sr-89, Sr-90; Sulfur-35; Technetium-99m; Thallium-201; Yttrium-90; and Ytterbium-164. In an embodiment, phosphorus 32 is alloyed into a steel endoprosthesis strut. In an embodiment, the beta-emitting isotope nickel 63 is used to provide the negative electric charge. Nickel 63 is an isotope with low energy beta emission and may be particularly useful in providing desire electric charge while allowing for a thinner insulating material. In an embodiment, the thickness of the endoluminal layer is between about 25 μm to about 70 μm. Variation in thickness of the active material will correspond to variable electric charge provided by the endoprosthesis 200. It should of course be understood that the internal layer 220 may also comprise any combination of metallic materials, polymers, any other biologically compatible materials and isotopes described herein, as long as the resulting material is capable of providing the target negative electric charge for a desired time period.

The external layer 250 may comprise one or more materials capable of providing the target positive electric charge, as described above, for a desired period of time. In an embodiment, the external layer 250 may be a metallic material, such as, by way of a non-limiting example, platinum, gold, copper, or any other metal with similar properties. It has been found that sputter-coated and ion bombardment deposition of copper with a coating thickness of about 20 μm may generate about +120 mV when exposed to blood electrolyte. By way of a non-limiting example, the external layer 250 may comprise a layer of copper of less than about 10 μm in thickness, with about 5 μm thickness likely to be sufficient to provide an electric charge of about +25 mV. It has also been shown that platinized or gold-pleated stents with a thickness of about 20 μm generate about +180 mV. In an embodiment, if platinum or gold are utilized in the external layer 250, the metal thickness may be less than about 5 μm thick to achieve the target positive charge. In another embodiment, the external layer may comprise a polymer providing positive electric charge, such as dimethylaminoethyl methacrylate, or any other biologically compatible material capable of providing the target positive electric charge. In yet another embodiment, the external layer 250 may comprise an isotope capable of providing the target positive electric charge. Such isotopes may be selected from isotopes that undergo beta-plus or alpha decay. It should of course be understood that the external layer 250 may also comprise any combination of metallic materials, polymers, any other biologically compatible materials and isotopes described herein, as long as the resulting material is capable of providing the target positive electric charge for a desired time period.

The one or more intermediate levels 230, 240 may comprise one or more insulating layers. Such one or more insulating layers may comprise an insulating polymer, ceramic, or any other material with suitable properties. The insulating layer may shield a vessel wall from the negative electric field. In an embodiment, the insulating layer is biologically inert, flexible to allow for stent expansion, and (in case of radioactive emitters being utilized as a source of negative electric charge) has a net 5 nCi or less of removable activity. Suitable insulating polymers include, but are not limited to, leaded acrylic, cyanoacrylates, ethylene methyl acrylate/acrylic acid, urethanes, thermal plastic urethane, saran polyvinylidene chloride, and other. In an embodiment, the thickness of the insulating layer may range from about 15 μm to about 30 μm, with less than 5 nCi of removable activity. Alternatively, the insulating layer may comprise a ceramic material. Such ceramic materials may include, but are not limited to, ceramics of alumina and glass-ceramics. The one or more intermediate layers may also comprise any other biologically compatible material or materials with insulating properties. In an embodiment, the one or more insulating layers may be designed, so as to contain negative electric field endoluminally, while containing the positive electric field exoluminally.

As noted above, the one or more intermediate layers may also include a structural layer. The structural layer properties are determined by the ability to be compressed, maintain desired geometry after inflation, corrosion resistance, and biologic inertness. Such structural layer may comprise steel or any other suitable material, materials or alloys based on titanium (such as nitinol, nickel titanium alloys, thermo-memory alloy materials), stainless steel, tantalum, nickel-chrome, cobalt-chromium or any similar materials. In an embodiment, the thickness of the intermediate structural layer ranges from about 13 μm to about 100 μm. In some embodiments, either the internal layer 220, external layer 250, or the one or more insulating material may serve in whole or in part as the structural layer. For example, the internal layer 220 may be constructed from a metal or alloy capable of providing the target negative charge and, at the same time, having adequate mechanical properties. In such embodiment, the structural layer may comprise in whole or in part the material of the internal layer 220.

In an embodiment, the endoprosthesis 200 includes four layers: the internal layer 220, the structural layer 230, the insulating material 240, and the external layer 250. The internal layer 220 may comprise an isotope, such as phosphorus 32, that can provide the negative electric charge of about −25 mV to about −200 mV for at least 6 weeks following the implantation of the endoprosthesis 200 into a body of a patient. The thickness of the internal layer 220 may range between about 25 μm and about 70 μm. The structural layer 240 may comprise a stent, such as a conventionally known stent. The thickness of the structural layer may vary between 100 μm and about 300 μm. The insulating layer may comprise an insulating polymer The thickness of the insulating material may vary between about 10 μm and about 50 μm. The external layer 250 may comprise a metal, such copper, coated over the stent. The thickness of the external layer may vary between about 5 μm and about 100 μm. It will of course be understood that the endoprosthesis 200 may comprise more than four layers or fewer than four layers, as desired.

In another embodiment, an inner surface of a conventionally known stent may be coated with nickel or silver to provide a negative point charge, whereas an outer surface of the stent may coated with gold or platinum to provide a positive point charge.

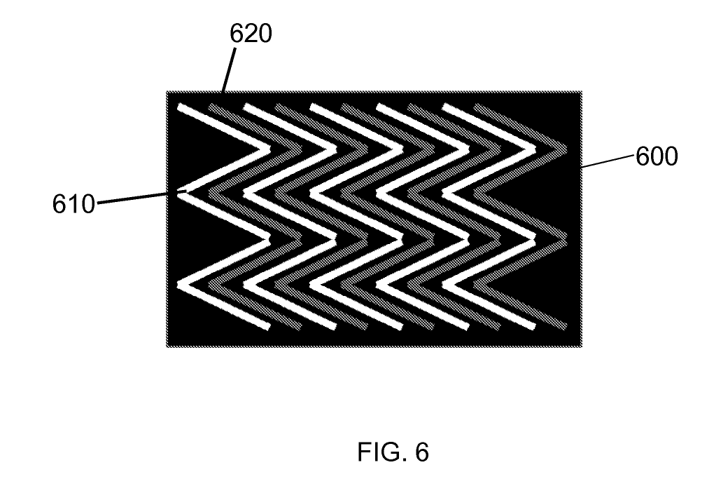

The endoprosthesis 200 may be manufactured using any conventional process for manufacturing similar devices. In an embodiment, the endoprosthesis 200 may be manufactured from a hollow tube structure using a conventional micro-machining technique and deposition process. Any hollow tube structure having adequate biological properties may be used including, but not limited to, stainless steel, gold, titanium, cobalt-chromium alloys, tantalum alloys, nitinol and various polymers. In an embodiment, the multiple layers described herein (including the internal layer, the external layer and an at least one intermediate layer) may be incorporated onto a conventional endoprosthesis. As illustrated in FIG. 6, in an embodiment, the endoprosthesis 200 may be manufactured from a hollow metal tube 600 which is cut in segments corresponding to a desired endoprosthesis length. The outer wall surface of the tube 600 may be covered with a layer of polymer (not visible) in excess of the thickness adequate for providing insulation between a negative electric charge material or emitter and a positive electric charge material or emitter. In an embodiment, the polymer covers the entire structural layer. In an embodiment, the polymer covers the endoluminal side of the structural layer. In an embodiment, the polymer covers the external side of the structural layer. The tube 600 is then cut to produce linear diagonal and/or longitudinal and/or curvilinear full-thickness openings 610 corresponding to a desired stent design. Any geometric pattern of the full-thickness openings 610 may be utilized. The resulting hollow tube 600 has exposed metal at the inner wall surface and at the sides of the openings 610 created by full-thickness cuts. In an embodiment, a desired pattern of full-thickness openings is laser cut into a tube. In an embodiment, a desired pattern of full-thickness openings is cut into a tube via chemical etching or electric discharge machining Electric charge material or emitter is then deposited onto the tube 600. The deposition process may take place before the cutting of the tube 600 openings 610, or after. In an embodiment, positively charged material is deposited first, followed by cutting of the openings 610, insulating material deposition, and negatively charged material deposition. In an embodiment, negatively charged material is deposited first, followed by cutting of the openings 610, insulating material deposition, and positively charged material deposition. In an embodiment, all materials are deposited on the structural layer before cutting of the openings 610.

The following description pertains to an embodiment where negatively charged material is applied first. A beta-emitting isotope, metal, polymer, or a combination thereof, providing sufficient negative electric charge, may be utilized. Techniques favoring preferential and secure material deposition onto metallic surfaces are utilized. Negative electric charge material or emitter is securely deposited onto exposed metal at the inner wall surface of the tube 600 and at the sides of the full-thickness openings 610. Excess of the negative electric charge material or emitter is then removed from the outer wall surface of the tube 600 utilizing light abrasion. Once the outer wall surface of the tube 600 is free of the negative electric charge material or emitter, indentations 620, complementing openings 610 are created at the outer wall polymer surface of the tube 600. It should be understood that the presented geometric patterns in FIG. 6 are for illustration purposes only and should not be construed as limiting.

The type and properties of the electric charge materials or radioactive emitters may be adjusted to ensure optimal duration and intensity of the negative and positive electric fields. Relative contributions of the negative and positive electric fields may be adjusted to ensure optimal biological functioning of an endoprosthesis of the present disclosure. Selection of electric charge materials or radioactive emitters allows the electric charge to be maintained for several years, aiming to reduce late endoprosthesis or stent thrombosis and secure endoprosthesis or stent anchoring. It is believed that the inward and side-ways negative electric field produced by an endoprosthesis of the present disclosure may counteract platelet aggregation, the important cause of stent thrombosis, and neointimal proliferation, the underlying cause of in-stent re-stenosis. It is also believed that the outward positive electric field produced by an endoprosthesis of the present disclosure may create an environment promoting secure anchoring of the endoprosthesis to a vessel wall. Using inward negative electric charges and outward positive electric charges has a potential to reduce stent thrombosis and in-stent re-stenosis while providing lasting secure anchoring of an endoprosthesis to the vessel wall. The complex physiology of an injured vessel response requires an endoprosthesis capable of producing electric fields of different polarity, intensity, and direction. In an embodiment, an inner endoluminal layer of an endoprosthesis of the present disclosure comprises a material or materials capable, when exposed to the blood interface, of maintaining a negative electric charge for 6 weeks or longer. Experience with endovascular stents indicates that 6 weeks is sufficient for an adequate endothelization of the implanted stent. In an embodiment, the target negative electric voltage is about −200 mV or below, however, other voltages may be desirable and used depending on the vessel diameter, lesion characteristic, arterial vs. venous location of the stent placement and other factors.

An additional insulating layer comprising polymer, ceramic, or any other suitable material may then be applied at the outer wall surface of the tube 600 with thickness allowing adequate depth of indentations 620 for positively charged material or emitter placement. In an embodiment, the insulating material is applied at the outer wall surface of the tube 600 using a chemical coating technique. In an embodiment, the insulating layer is an adhesive layer that adheres to the outer wall surface of the tube 600. An alpha-emitting isotope, metal, polymer, or a combination thereof, providing sufficient positive electric charge, may be utilized. Positively charged material or emitter is then deposited onto the outside surface of the tube 600. Techniques favoring secure deposition of the positively charged material or emitter to the polymer are utilized at this stage. The positively charged material or emitter can be deposited directly onto the surface of the polymer or an adhesive material can be applied first, followed by the deposition of the positively charged material. Excess of the charged material or emitter is then removed, for example with light abrasion, allowing sufficient amount of the material to be retained in the indentations 620 and preserving insulating layer outside of the indentations.

An endoprosthesis of the present disclosure can be used for the treatment of a variety of disorders, including, but not limited to, atherosclerotic vascular obstructions, vascular aneurysms, vascular dissections, vascular in-stent stenosis, and vascular in-stent thrombosis. An endoprosthesis of the present disclosure can be used to treat the following conditions that result from blocked or damaged blood vessels: coronary heart disease (CHD) (angioplasty and stent placement—heart); peripheral artery disease (angioplasty and stent replacement—peripheral arteries); renal artery stenosis; abdominal aortic aneurysm (aortic aneurysm repair—endovascular); and carotid artery disease (carotid artery surgery). Other reasons to use an endoprosthesis of the present disclosure includes: keeping open a blocked or damage ureter (percutaneous urinary procedures); treating aneurysms, including thoracic aortic aneurysms; keeping bile flowing in blocked bile ducts (biliary stricture); and helping a patient breathe if they have a blockage in the airways. An endoprosthesis of the present disclosure may be utilized for treatment of many conditions including, but not limited to, coronary artery disease, cerebrovascular disease, peripheral vascular disease, Kawasaki disease, coronary dissections, peripheral vascular dissections, aortic aneurysms, and aortic dissections.

An endoprosthesis of the present disclosure can be of any length or diameter depending on the intended use. An endoprosthesis of the present disclosure can be mounted on an intravascular catheter for percutaneous placement into a vessel. An endoprosthesis of the present disclosure can be placed in the vessel percutaneously or surgically through vessel dissection. An endoprosthesis of the present disclosure can be self-expandable or require balloon inflation to achieve contact with the vessel or vascular cavity wall.

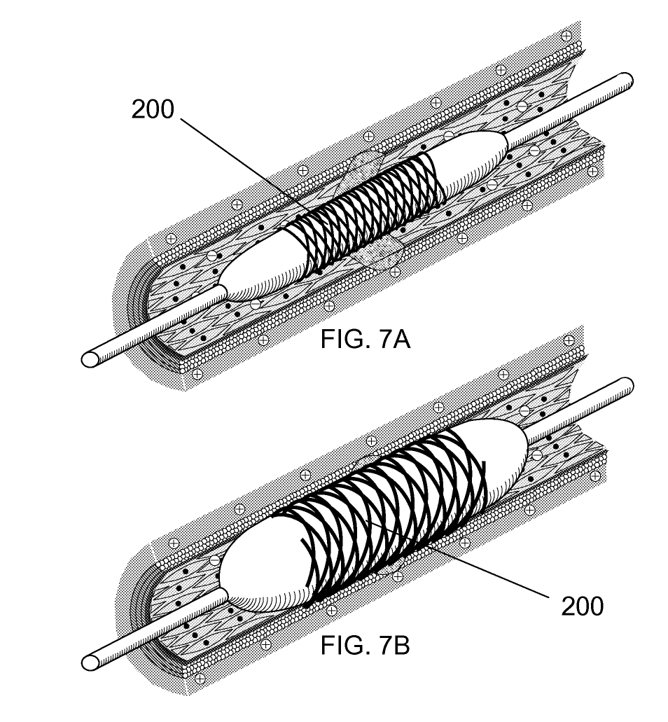

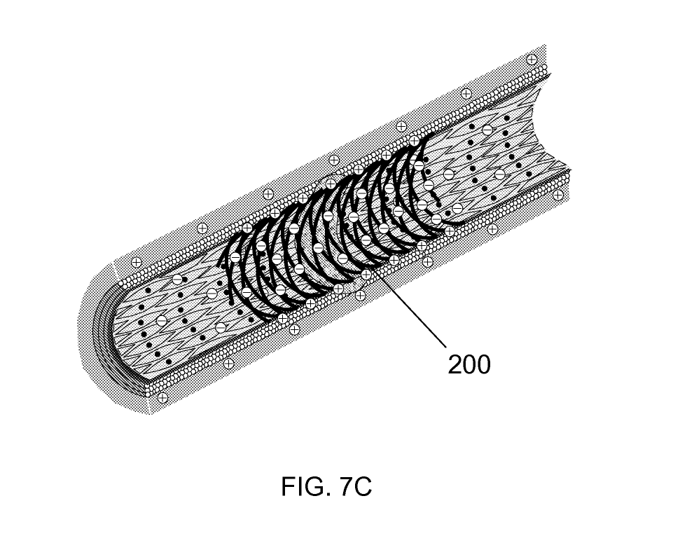

In an embodiment, an endoprosthesis of the present disclosure can be implanted during a percutaneous coronary intervention (PCI) procedure, also known as angioplasty, as illustrated in FIGS. 7A, 7B and 7C. In an embodiment, a method of performing a percutaneous coronary intervention procedure to implant an endoprosthesis of the present disclosure includes obtaining intravascular access through the femoral or the brachial artery; advancing a first wire proximally until the first wire reaches the ascending aorta; advancing a hollow catheter over the first wire; positioning the hollow catheter at a desired coronary ostium; withdrawing the first wire; advancing a guidewire through the hollow catheter into a blocked artery; positioning the guidewire across the blocked portion of the artery; withdrawing the hollow catheter; advancing a delivery catheter with the endoprosthesis over the blocked portion of the artery, as shown in FIG. 7A; confirming correct positioning of the endoprosthesis; expanding the endoprosthesis inside the artery, as shown in FIG. 7B, and removing the delivery catheter, as shown in FIG. 7C. Following the removal of the catheter, hemostasis may be achieved either with manual compression or with sealing devices.

In another embodiment, a method of performing a percutaneous coronary intervention procedure to implant an endoprosthesis of the present disclosure includes threading a catheter from the groin area of a patient into a blocked vessel having plaque; opening the blocked vessel using balloon angioplasty which compresses the plaque against walls of the blocked vessel; flattening the plaque so that blood can flow through the vessel freely; deploying the endoprosthesis to push against the wall of the artery to keep the wall of the artery open.

Alternatively, an endoprosthesis of the present disclosure may be surgically placed into a lumen. In an embodiment, a method of placing an endoprosthesis of the present disclosure includes dissecting skin, subcutaneous tissue, and lumen wall of a patient; and placing the endoprosthesis into the lumen. Catheters and guidewires may be utilized if the desired area of lumen requiring treatment cannot be readily reached through direct lumen wall dissection.

After placement of the endoprosthesis into a lumen, the internal layer 220 is exposed to a bodily fluid, and thus generates negative electric field, preponderance of which may be directed by the insulating layer(s) inward and side-ways. Through electrostatic forces negatively charged coagulation proteins and blood cells are repelled from the negatively charged internal layer of the endoprosthesis. The external layer generates positive electric field. Positive electric filed in vicinity of lumen tissue attracts cell migration and neointimal formation, which may aid in securing anchoring of the endoprosthesis 200. These cell migration and neointimal formation over the course of 6 weeks or less may also help endothelization of the endoprosthesis 200. In the presence of negative electric field directed into the lumen of the vessel, endothelization may be delayed beyond 6 weeks. However, due to the negative electric field generated by the endoprosthesis 200, thrombus formation on the inner surface of the endoprosthesis may be avoided.

In an embodiment, an endoprosthesis of the present disclosure includes an internal layer designed to provide a negative electric field directed endoluminally; an external layer designed to provide a positive electric field directed exoluminally; and one or more intermediate layers disposed between the internal layer and the external layer, wherein the negative electric field is due to a negative point charge between about −25 mV and about −250 mV, and wherein the positive electric field is due to a positive point charge between about +1 mV and about +30 mV.

In an embodiment, an endoprosthesis of the present disclosure includes a plurality of struts, wherein each strut has an internal layer, an external layer, and one or more intermediate layers therebetween, wherein the internal layer includes a material that provides a negative electric field directed endoluminally, wherein the external layer includes a material that provides a positive electric field directed exoluminally, and wherein the one or more intermediate layers include a material that provides an insulation between the internal layer and the external layer.

In an embodiment, a method of treating a blood vessel is provided. The method includes the steps of: deploying an endoprosthesis inside the blood vessel, the endoprosthesis comprising: an internal layer designed to provide a negative electric field directed endoluminally; an external layer designed to provide a positive electric field directed exoluminally; and one or more intermediate layers disposed between the internal layer and the external layer, wherein the negative electric field is created by a negative point charge between about −25 mV and about −250 mV, and wherein the positive electric field is created by a positive point charge between about +1 mV and about +30 mV so as to treat the blood vessel.

All patents, patent applications, and published references cited herein are hereby incorporated by reference in their entirety. While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains, and as fall within the scope of the appended claims.

What is claimed is:

1. An endoprosthesis comprising:
an internal layer designed to provide a negative electric field directed endoluminally;
an external layer designed to provide a positive electric field directed exoluminally; and
one or more intermediate layers disposed between the internal layer and the external layer,
wherein the negative electric field is due to a negative point charge between about −25 mV and about −250 mV, and
wherein the positive electric field is due to a positive point charge between about +1 mV and about +30 mV.

2. The endoprosthesis of claim 1, wherein the negative electric field is directed endoluminally in both a radial direction and a longitudinal direction.

3. The endoprosthesis of claim 1, wherein the negative electric field is dispersed at an angle more than about 180 degrees.

4. The endoprosthesis of claim 1, wherein the negative electric field blankets an inside surface of the endoprosthesis substantially in its entirety.

5. The endoprosthesis of claim 1, wherein the positive electric field is directed exoluminally in a radial direction.

6. The endoprosthesis of claim 1, wherein the positive electric field is dispersed at an angle less than about 120 degrees.

7. The endoprosthesis of claim 1, wherein the material that provides the negative point charge is capable of maintaining the negative point charge for a period of at least six weeks.

8. The endoprosthesis of claim 1, wherein the internal layer comprises a metal capable of providing a charge between about −150 mV and about −250 mV.

9. The endoprosthesis of claim 1, wherein the internal layer comprises an isotope capable of providing a charge between about −25 mV and about −200 mV.

10. The endoprosthesis of claim 1, wherein the external layer comprises a metal capable of providing a charge between about +1 mV and about +30 mV.

11. An endoprosthesis comprising:
a plurality of struts, wherein each strut has an internal layer, an external layer, and one or more intermediate layers therebetween,
wherein the internal layer includes a material that provides a negative electric field directed endoluminally,
wherein the external layer includes a material that provides a positive electric field directed exoluminally, and
wherein the one or more intermediate layers include a material that provides an insulation between the internal layer and the external layer.

12. The endoprosthesis of claim 11, wherein the negative electric field is created by a negative point charge between about −150 mV and about −250 mV.

13. The endoprosthesis of claim 11, wherein the positive electric field is created by a positive point charge between about +1 mV and about +30 mV.

14. The endoprosthesis of claim 11, wherein the struts are designed such that the negative electric field is dispersed at an angle less than about 180 degrees

15. The endoprosthesis of claim 11, wherein the struts are designed such that the negative electric field blankets an inside surface of the endoprosthesis substantially in its entirety.

16. The endoprosthesis of claim 11, wherein the struts are designed such that the positive electric field is directed in a radial direction.

17. The endoprosthesis of claim 11, wherein the struts are designed such that the positive electric field is dispersed at an angle less than about 120 degrees.

18. A method of treating a blood vessel comprising:
deploying an endoprosthesis inside the blood vessel, the endoprosthesis comprising:
an internal layer designed to provide a negative electric field directed endoluminally;
an external layer designed to provide a positive electric field directed exoluminally; and
one or more intermediate layers disposed between the internal layer and the external layer,
wherein the negative electric field is created by a negative point charge between about −25 mV and about −250 mV, and
wherein the positive electric field is created by a positive point charge between about +1 mV and about +30 mV so as to treat the blood vessel.

19. The method of claim 18, wherein the one or more intermediate layers comprises a stent.

20. The method of claim 19, wherein the one or more intermediate layers include a material that provides an insulation between the internal layer and the external layer
